(19) United States
(12) Patent Application Publication
(10) Pub. No.: US 2026/0055397 A1
(43) Pub. Date: Feb. 26, 2026

(54) NOVEL METHOD FOR SIZE SELECTING NUCLEIC ACIDS

(71) Applicant: Genome Research Limited, Cambridge (GB)

(72) Inventor: Naomi PARK, Cambridge (GB)

(21) Appl. No.: 19/102,614

(22) PCT Filed: Aug. 11, 2023
(86) PCT No.: PCT/GB2023/052127

§ 371 Date: Feb. 10, 2025

(30) Foreign Application Priority Data
Aug. 11, 2022 (GB) 2211766.7

(51) Int. Cl. C12N 15/10

(52) U.S. Cl. CPC C12N 15/1068

(57) ABSTRACT

The invention relates to a method of preparing a nucleic acid library for sequencing comprising selecting for nucleic acid fragments greater than 5 kb in length from a fragmented nucleic acid sample using paramagnetic Solid Phase Reversible Immobilization (SPRI) beads in a binding buffer. The binding buffer comprises specified concentrations of PEG6000, NaCl and TrisHCl at pH8, and the ratio of SPRI beads in binding buffer to sample is between 0.97× and 0.91×. Also provided are methods of sequencing the genome of an organism and generating novel genome assemblies.

### FIELD OF THE INVENTION

The invention relates to a method of preparing a nucleic acid library for sequencing comprising selecting for nucleic acid fragments greater than 5 kb in length from a fragmented nucleic acid sample using paramagnetic Solid Phase Reversible Immobilization (SPRI) beads in a binding buffer. The binding buffer comprises specified concentrations of PEG6000, NaCl and TrisHCl at pH8, and the ratio of SPRI beads in binding buffer to sample is between 0.97× and 0.91×. Also provided are methods of sequencing the genome of an organism and generating novel genome assemblies.

### BACKGROUND OF THE INVENTION

Long-read sequencing technologies are essential for maximising the generation of high-quality genome assemblies from a single specimen (Rhie et al. (2021) Nature, 592(7856): 737-746. In order to generate sufficient coverage for genome assembly, it is critical to maximise yields of high-quality long-read data derived from often limited amounts of DNA. Recent advances in library preparation protocols enable a reduction in mass of DNA input requirements, putting long-read based assemblies in reach for small highly heterozygous organisms that comprise much of the diversity of life (Kingan et al. (2019) Genes (Basel), 10(1):62. Nonetheless, high-throughput methodologies for DNA extraction from a diverse sample type and size frequently yields limited amounts of DNA of which a significant proportion contains a size distribution below that which is optimal for long-read sequencing.

One such method of long-read sequencing, Pacbio (instruments include RSII, Sequel, and Sequel II), proceeds via the detection of fluorescence events. Such events are detected that correspond to the addition of one specific nucleotide by a polymerase tethered to the bottom of a ZMW well. High fidelity (HiFi) reads are a data type generated via a circular consensus sequencing (CCS) mode. This mode provides base-level resolution with >99.9% single-molecule read accuracy. In order for the polymerase to have sufficient passes through the same template molecule for CCS, the desirable input target fragment length is ˜15-18 kb (https://www.pacb.com/wp-content/uploads/Procedure-checklist-Preparing-whole-genome-and-metagenome-libraries-using-SMRTbell-prep-kit-3.0.pdf). Shorter fragments, whilst generating high-quality data, inhabit ZMW real estate for the duration of the run and reduce HiFi data yield.

Therefore, where DNA extractions generate a smear of shorter fragments, a method for their removal improves sequencing yield outcomes. Whilst techniques such as gel size selection are highly effective (https://www.pacb.com/wp-content/uploads/Technical-note-Alternative-size-selection-methods-for-SMRTbell-ep-kit-3.0.pdf), requisite input amounts often exceed that available. Additionally, the resultant post-size selection yield may be insufficient for onwards progression to sequencing. Other, bead-based, commercial methods for size selection have cutoffs (100-1000 bp) which are of limited effectiveness for long read-sequencing.

Accordingly, there remains a desire for a technology for size selection >10 kb that is tolerant to a wide range of input amounts, does not add complexity to laboratory workflows, is amenable to automation and reliably recovers high yields of on target library fragments.

### SUMMARY OF THE INVENTION

According to a first aspect of the invention, there is provided a method of generating a nucleic acid library for sequencing, said method comprising selecting for nucleic acid fragments greater than 5 kb in length from a fragmented nucleic acid sample using paramagnetic Solid Phase Reversible Immobilization (SPRI) beads in a binding buffer comprising: 10% PEG6000; 1900 mM NaCl; and 10 mM TrisHCl pH8,
wherein the ratio of SPRI beads in binding buffer to sample is between 0.97× and 0.91×.

According to another aspect, there is provided a method of sequencing the genome of an organism, comprising performing the method described herein to generate a nucleic acid library. In one embodiment, the genome is a novel genome. In a further embodiment, the method of sequencing a genome is for generating a novel genome assembly.

In a further aspect of the invention, there is provided the use of the methods described herein for generating a novel genome assembly.

### BRIEF DESCRIPTION OF THE FIGURES

FIG. 1: Size selection of nucleic acid fragments. Extracted gDNA of a profile with a smear of material <10 kb was analysed before (top panel) and after (bottom panel) selection using SPRI beads in binding buffer at the indicated ratios as described herein.

FIG. 2: A) Size analysis of sheared gDNA from mouse subjected to the selection of nucleic acid fragments of a specified size using SPRI beads in binding buffer at the indicated ratios as described herein. B) Read length distribution analysis following sequencing of mouse gDNA nucleic acid library generated by the method described herein.

FIG. 3: A) Size analysis of sheared gDNA from mistletoe taken through Pacbio SPK.3.0 kit library construction with (“x0.97 Modified SPRI”) or without (“No Modified SPRI”) being subjected to the selection of nucleic acid fragments of a specified size as described herein. B) Read length distribution analysis following sequencing of mistletoe gDNA nucleic acid library generated by the method described herein.

FIG. 4: A) Size analysis of sheared gDNA from green algae taken through Pacbio SPK.3.0 kit library construction with (“x0.97 Modified SPRI”) or without (“No Modified SPRI”) being subjected to the selection of nucleic acid fragments of a specified size as described herein. B) Read length distribution analysis following sequencing of green algae gDNA nucleic acid library generated either with (bottom panel) or without (top panel) size selection as described herein.

FIG. 5: A) Size analysis of sheared gDNA from snail taken through Pacbio SPK.3.0 kit library construction with (“x0.97 Modified SPRI”) or without (“No Modified SPRI”) being subjected to the selection of nucleic acid fragments of a specified size as described herein. B) Read length distribution analysis following sequencing of snail gDNA nucleic acid library generated either with (bottom panel) or without (top panel) size selection as described herein.

FIG. 6: A) Size analysis of sheared gDNA from ladybird taken through Pacbio SPK.3.0 kit library construction with (“x0.97 Modified SPRI”) or without (“No Modified SPRI”) being subjected to the selection of nucleic acid fragments of a specified size as described herein. B) Read length distribution analysis following sequencing of ladybird gDNA nucleic acid library generated either with (bottom panel) or without (top panel) size selection as described herein.

FIG. 7: A) Size analysis of sheared gDNA from oak taken through Pacbio SPK.3.0 kit library construction with (“x0.97 Modified SPRI”) or without (“No Modified SPRI”) being subjected to the selection of nucleic acid fragments of a specified size as described herein. B) Read length distribution analysis following sequencing of oak gDNA nucleic acid library generated either with (bottom panel) or without (top panel) size selection as described herein.

### DETAILED DESCRIPTION OF THE INVENTION

According to a first aspect of the invention, there is provided a method of generating a nucleic acid library for sequencing, said method comprising selecting for nucleic acid fragments greater than 5 kb in length from a fragmented nucleic acid sample using paramagnetic Solid Phase Reversible Immobilization (SPRI) beads in a binding buffer comprising: 10% PEG6000; 1900 mM NaCl; and 10 mM TrisHCl pH8,
wherein the ratio of SPRI beads in binding buffer to sample is between 0.97× and 0.91×.

The terms “select”, “selecting” and “selection” which are used interchangeably herein, refer to the enrichment of nucleic acid fragments of certain lengths from a fragmented nucleic acid sample, such as by preferential isolation, while those either below or above the specified length (e.g. fragments which are shorter or longer than the specified cut-off length) are removed from the fragmented nucleic acid sample. Following selection, the nucleic acid library will therefore comprise nucleic acid fragments of the specified length (e.g. fragments greater than 5 kb and/or 7.5 kb in length) and will not substantially comprise fragments having lengths below said specified length, i.e. those fragments which are shorter than the specified length will be absent/removed. In particular, following selection the nucleic acid library will not substantially comprise fragments below the specified length, i.e. those fragments which are shorter than the specified length will be absent/removed. Thus, disclosed herein is a method for generating a nucleic acid library for sequencing which comprises the removal of nucleic acid fragments 5 kb or less in length from a fragmented nucleic acid sample. As such, in one embodiment, selecting nucleic acid fragments removes nucleic acid fragments 5 kb or less in length from the fragmented nucleic acid sample. In a certain embodiment, selecting nucleic acid fragments removes nucleic acid fragments less than 5 kb in length. In further embodiments, the selected nucleic acid fragments are greater than or equal to 5.5 kb, 6 kb, 6.5 kb or 7 kb in length. Thus, in some embodiments, selecting nucleic acid fragments removes nucleic acid fragments 5.5 kb or less, 6 kb or less, 6.5 kb or less or 7 kb or less in length from the fragmented nucleic acid sample. In another embodiment, the selected nucleic acid fragments are greater than or equal to 7.5 kb in length, such as greater than 7.5 kb in length. In a further embodiment, selecting nucleic acid fragments removes nucleic acid fragments 7.5 kb or less in length from the fragmented nucleic acid sample. In a yet further embodiment, selecting nucleic acid fragments removes nucleic acid fragments less than 7.5 kb in length. In still further embodiments, the selected nucleic acid fragments are greater than or equal to 8 kb, 8.5 kb, 9 kb or 9.5 kb in length. Thus, in some embodiments, selecting nucleic acid fragments removes nucleic acid fragments 8 kb or less, 8.5 kb or less, 9 kb or less or 9.5 kb or less in length from the fragmented nucleic acid sample. In a yet other embodiment, the selected nucleic acid fragments are greater than or equal to 10 kb in length. Thus, in a further embodiment, selecting nucleic acid fragments removes nucleic acid fragments 10 kb or less in length from the fragmented nucleic acid sample. In a certain embodiment, selecting nucleic acid fragments removes nucleic acid fragments less than 10 kb in length. In yet further embodiments, the selected nucleic acid fragments are greater than or equal to 11 kb, 12 kb, 13 kb, 14 kb or 15 kb in length.

Nucleic acid libraries generated by selecting for nucleic acid fragments of a specified length are useful in sequencing applications where data for long sequence read lengths (e.g. >5 kb, such as ≥7.5 kb, such as ≥10 kb) or the filtering out/removal of sequencing reads for short sequences (e.g. ≤7.5 kb, such as ≤5 kb) is desired. Such applications include, but are not limited to, the sequencing of whole genomes, such as for the generation of novel genome assemblies. Thus, according to one aspect of the present invention, there is provided a method of sequencing the genome of an organism, comprising performing the method described herein to generate a nucleic acid library. In one embodiment, the genome is a novel genome. Thus, in a further embodiment, the method of sequencing a genome is for generating a novel genome assembly. In a further aspect of the invention, there is provided the use of the methods described herein for generating a novel genome assembly. Selecting nucleic acid fragments of a specified length (e.g. >5 kb, such as ≥7.5 kb, such as ≥10 kb) or removing fragments shorter or longer (in particular, shorter) than the specified length ensures that sequencing read data is focused on the nucleic acid fragments of interest, allowing greater read depth for said nucleic acid fragments. For example, in the context of Pacbio CCS, fragments shorter than the specified length can occupy a ZMW for the duration of the run and reduce HiFi data yield. Furthermore, when sequencing whole genomes, longer sequencing reads are desirable to minimise the need for processing of the sequencing data and computational alignment of reads or removal of duplicated reads (which have been introduced, for example, during preparation of the library for sequencing). This is especially useful when sequencing large repeat or low complexity regions which are often refractory to other sequencing methodologies. Thus, in certain embodiments, the nucleic acid fragment length is the N50 length. N50 is the shortest sequencing read length (or contig length—contiguous fragments of DNA sequence) that needs to be included for covering 50% of the nucleic acid sample, such as a genome comprised in the sample, i.e. half of the genome sequence is covered by reads larger than or equal the N50 size and the sum of the lengths of all reads of size N50 or longer contain at least 50% of the total genome sequence.

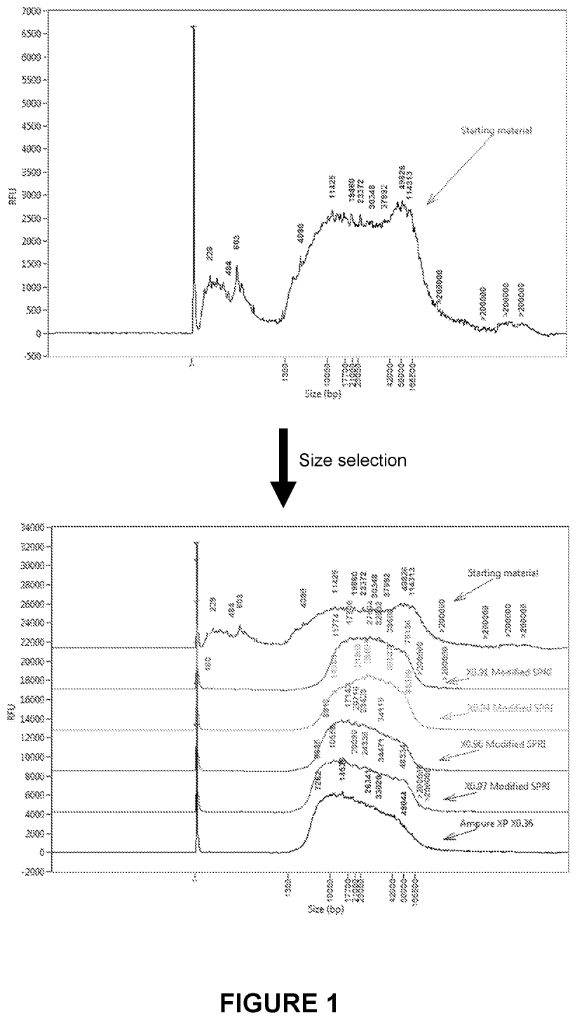

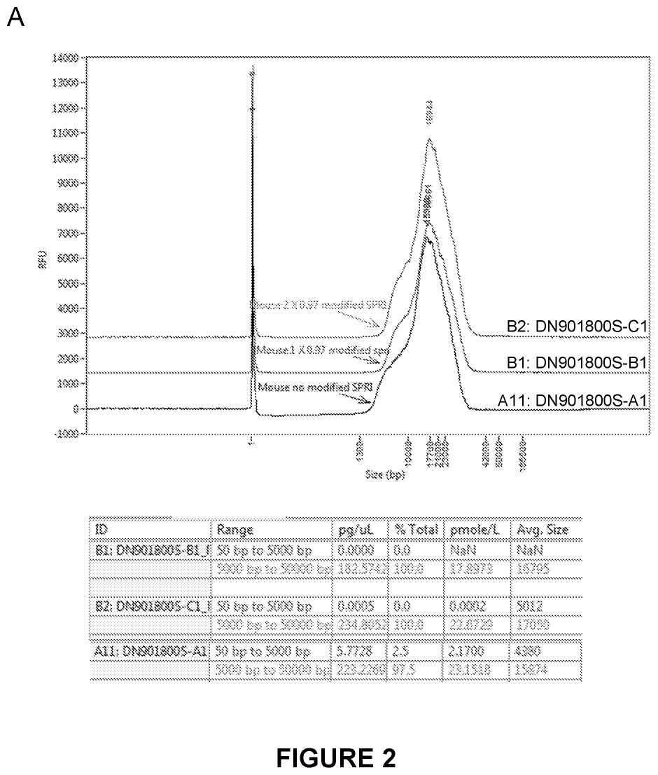

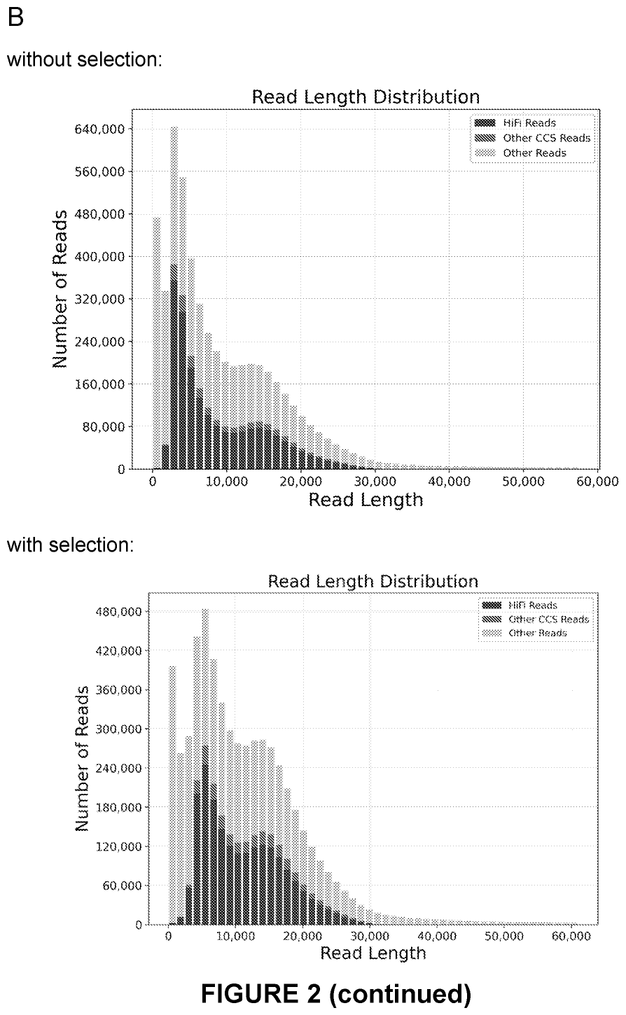

Other methods of generating nucleic acid libraries for sequencing comprising the selection of nucleic acid fragments of specified lengths using SPRI beads are known in the art and include the use of, for example, AMPure XP or SPRIselect beads from Beckman Coulter (https://www.mybeckman.uk/reagents/genomic/cleanup-and-size-selection) and ProNex (Promega), Mag-Bind TotalPure (Omega BioTek), NucleoMag NGS Clean-up and Size Select (Fisher Scientific), MagSi-NGS^{PREP} Plus (Magtivio), KAPA Pure and KAPA HyperPure Beads (Roche), PCRCleanDX and DNA SizeSelector (Aline Biosciences), Sera-Mag Select (Cytiva) and AmpliClean Cleanup kit (NimaGen). Non-commercial protocols for selecting specified nucleic acid fragment lengths are also known and include both the use of SPRI beads and the direct precipitation of nucleic acid fragments from solution without beads. Examples of protocols which directly precipitate nucleic acid fragments (i.e. which do not use beads) use polyethylene glycol (PEG; such as PEG6000 (PEG6k), PEG8000 (PEG8k) or PEG 20000 (PEG20k)) or polyvinylpyrrolidone (PVP; such as PVP 360000) in combination with salt (NaCl or KCl) to select nucleic acid fragments of various lengths (e.g. by varying PEG size and concentration or PVP concentration with different amounts of salt). In particular, Tyson, J (2020), protocols.io (https://dx.doi.org/10.17504/protocols.io.7erhid6 and https://www.longreadclub.org/mountain-protocol/) uses 4.5-7.5% PEG6k, PEG8k or PEG20k in various combinations with either 400 mM-1 M KCI or 260-630 mM NaCl to select large/long nucleic acid fragments, while in Jones et al. (2021), protocols.io (dx.doi.org/10.17504/protocols.io.bwr8pd9w) direct precipitation with 3% or 4% PVP 360000 and 1.2 M KCl or 9% PEG8k and 1 M NaCl is used to select nucleic acid fragments >10 kb or >25 kb in length. Protocols using SPRI beads include Schalamun & Schwessinger (2017), protocols.io (https://dx.doi.org/10.17504/protocols.io.idmca46) which uses a binding buffer comprising 4.8-5.5% PEG8k and 0.7-0.8 M NaCl to select nucleic acid fragments >2 kb in length (i.e. fragments below 1-2 kb are removed), and Stortchevoi et al. (2020), J. Biomol. Tech. (https://dx.doi.org/10.7171%2Fibt.20-3101-002) which uses a binding buffer comprising 5% PEG8k and 500 mM MgCl₂ to select nucleic acid fragments ≥10 kb in length or a binding buffer comprising 20% PEG8k and 750 mM NaCl to select nucleic acid fragments ≥6 kb in length. However, these methods developed to date either do not provide selection of large nucleic acid fragments (such as >7.5 kb in length; e.g. as in Schalamun & Schwessinger (2017)) or provide a low recovery of selected nucleic acid fragments (e.g. Stortchevoi et al. (2020) achieves only 25% recovery of selected fragments ≥10 kb and 36-45% recovery of selected fragments ≥7.7 kb; see FIGS. 1A and 2B therein). Furthermore, in Stortchevoi et al. (2020) for example, it is reported that the use of a binding buffer comprising 5% PEG8k with monovalent salts (i.e. other than MgCl₂) leads to the complete loss of nucleic acid fragment immobilisation on SPRI beads, and very large fragments >42 kb in length are lost. Methods which do not use beads (e.g. Tyson, J (2020)) often require a large amount of starting sample in order to directly precipitate DNA from solution.

By contrast, in some embodiments, the nucleic acid library generated by the method described herein comprises very large nucleic acid fragments, such as those longer/greater than 42 kb in length, i.e. very large nucleic acid fragments present in the fragmented nucleic acid sample are retained in the nucleic acid library. Thus, in one embodiment, the generated nucleic acid library comprises nucleic acid fragments greater than 42 kb in length. In further embodiments, the recovery of selected nucleic acid fragments in the nucleic acid library generated by the method described herein is about 32%, 44%, 73%, 92%, 82%, 51-100% or 42-100% of the nucleic acid fragments of the specified length in the fragmented nucleic acid sample. In other embodiments, the recovery of selected nucleic acid fragments is dependent on the amount of nucleic acid fragments below/shorter than the specified length present in the fragmented nucleic acid sample. In one embodiment, the recovery of selected nucleic acid fragments is greater than 30%, such as greater than 40%, greater than 50%, greater than 60%, greater than 70%, greater than 80%, greater than 90% or greater than 95% of the nucleic acid fragments of the specified length in the fragmented nucleic acid sample. In another embodiment, the recovery of selected nucleic acid fragments is 100%. In a further embodiment, the recovery of selected nucleic acid fragments (e.g. greater than 5 kb in length) is greater than 30%, such as greater than 40%, greater than 50%, greater than 60%, greater than 70%, greater than 80%, greater than 90% or greater than 95% of the nucleic acid fragments greater than 5 kb in length in the fragmented nucleic acid sample. In a yet further embodiment, the recovery of selected nucleic acid fragments greater than 5 kb in length is about 100%. In another embodiment, the recovery of selected nucleic acid fragments is greater than 30%, such as greater than 40%, greater than 50%, greater than 60%, greater than 70%, greater than 80%, greater than 90% or greater than 95%. In a still further embodiment, the recovery of selected nucleic acid fragments (e.g. greater than 7.5 kb in length) is greater than 30%, such as greater than 40%, greater than 50%, greater than 60%, greater than 70%, greater than 80%, greater than 90% or greater than 95% of the nucleic acid fragments greater than 7.5 kb in length in the fragmented nucleic acid sample. In a particular embodiment, the recovery of selected nucleic acid fragments greater than 7.5 kb in length is greater than 70%. In a further embodiment, the recovery of selected nucleic acid fragments greater than 7.5 kb in length is greater than 90%. In a yet further embodiment, the recovery of selected nucleic acid fragments greater than 7.5 kb in length (e.g. fragments about 8 kb or greater in length) is greater than 40%. In a still further embodiment, the recovery of selected nucleic acid fragments greater than 7.5 kb in length (e.g. fragments about 9 kb or greater in length) is greater than 30%. In a further embodiment, the recovery of selected nucleic acid fragments greater than 7.5 kb in length is about 100%.

The terms “nucleic acid sample” and “sample” as used herein refer to any composition comprising nucleic acid, including genomic DNA (gDNA), sequence-selected nucleic acids, epigenetically modified nucleic acids, RNA and/or transcriptomic cDNA. Nucleic acid samples comprising gDNA allow the sequencing of whole genomes, such as for the generation of novel genome assemblies. Sequence-selected nucleic acids include nucleic acids which are enriched for a genomic region of interest, such as specific genes or a panel of specific genes, coding sequences (CDSs; e.g. protein coding sequences), exonic sequences (including the whole exome) and intronic sequences, and may be selected using any suitable method known in the art, such as using oligonucleotide probes having sequences which are complementary to the genomic region(s) of interest. Nucleic acid samples comprising RNA and/or transcriptomic cDNA allow the transcriptome of a cell, tissue/organ or organism to be evaluated, including gene expression levels, transcript usage, splicing events, isoform identification and the identification of gene structures, regulatory elements and coding regions. Thus, in one embodiment, the nucleic acid sample is obtained from a cell, such as a single cell (e.g. an in vitro cultured cell or in vitro cultured cells). In another embodiment, the nucleic acid sample is obtained from ex vivo cells, such as a single ex vivo cell. In a further embodiment, the nucleic acid sample is obtained from a cell nucleus. In an alternative embodiment, the nucleic acid sample is obtained from the cytosol of a cell, such as the mRNA outside of the cell nucleus (i.e. the nucleic acid sample is derived from the transcriptome of the cell). In a yet further embodiment, the nucleic acid sample is obtained from a tissue or organ. In a still further embodiment, the nucleic acid sample is obtained from a whole organism. In some embodiments, the cell, tissue or organ is from an animal or plant, or the organism is an animal or plant. In one embodiment, the animal or plant is selected from the group including, but not limited to, invertebrates (e.g. worms and insects), mammals, fungi, birds, reptiles, amphibians, fish and plants. In a particular embodiment, the nucleic acid sample is obtained from a plant or animal for which the genome sequence is not known or is incomplete. Thus, in a further embodiment, the fragmented nucleic acid sample comprises genomic DNA (gDNA).

The terms “fragment”, “fragments” and “fragmented” as used herein, refer to any nucleic acid sequence that is shorter than the sequence from which it is derived, or to the process of creating said shorter nucleic acid sequences. For example, the nucleic acid sample may comprise gDNA which is fragmented into fragments of suitable size/length for sequencing, such as about 30 kb, but can be of any size, ranging from several megabases (mb) and/or kilobases (kb) to only a few nucleotides long. The nucleic acid sample may be fragmented using any method known in the art, including but not limited to, mechanical fragmentation (e.g. by sonication or shearing, such as hydrodynamic shearing) and/or enzymatic digestion (e.g. using an exonuclease or combination of exonuclease enzymes). In one embodiment, the nucleic acid sample is fragmented by shearing. In another embodiment, fragmentation is by sonication. In a further embodiment, the nucleic acid sample is fragmented by shearing to fragments with an average length of about 30 kb or greater. In a yet further embodiment, fragmentation by shearing comprises passing the nucleic acid sample through a needle or pipette tip, such as a small-bore pipette tip. In one embodiment, fragmentation by shearing comprises passing the nucleic acid sample through a 29 gauge (29G) needle (i.e. a needle having a nominal internal diameter of 0.184 mm). In an alternative embodiment, fragmentation by shearing comprises passing the nucleic acid sample through a 26 gauge (26G) needle (i.e. having a nominal internal diameter of 0.26 mm). In another embodiment, fragmentation by shearing comprises passing the nucleic acid sample through a 21 gauge (21G) needle (i.e. having a nominal internal diameter of 0.514 mm).

### SPRI Beads

Solid Phase Reversible Immobilization (SPRI) beads are known in the art and are common in several commercial and non-commercial kits/protocols for purifying nucleic acids, such as from PCR reaction mixtures. They are paramagnetic (i.e. they are only magnetic when subjected to a magnetic field) which prevents clumping and precipitation during storage and use, and are made of polystyrene surrounded by a layer of magnetite coated with carboxyl molecules. The surface coating of carboxyl molecules reversibly binds to nucleic acids such as DNA in the presence of a crowding agent, such as polyethylene glycol (PEG) or polyvinylpyrrolidone (PVP) and a salt, in the binding buffer. The crowding agent reduces the effective volume of solute in the binding buffer, forcing the nucleic acid and beads into close proximity/contact and causing the negatively charged nucleic acid molecules to bind to the carboxyl groups, immobilising the nucleic acid molecules on the bead surface. The immobilisation is dependent on the concentration of crowding agent and salt in the binding buffer, so the volumetric ratio of beads in binding buffer to nucleic acid sample is critical. Following immobilisation/binding of nucleic acid molecules on the SPRI beads, a magnetic field is applied to the sample to pull the bead/nucleic acid complexes (also known as microparticles) out of solution and separate them from the remaining sample mixture which remains in solution. Following one or more wash in the presence of the magnetic field (e.g. using ethanol), the magnetic field is removed and an aqueous elution buffer is added to elute the nucleic acid molecules off the beads and into solution. Any suitable elution buffer may be used, such as 10 mM Tris-HCl or a proprietary elution buffer. SPRI beads are easy to handle and their binding capacity for nucleic acids is very large. For example, 1 μl of AMPure XP beads (Beckman Coulter) will bind over 3 μg of DNA. As well as for purification of nucleic acids, SPRI beads are known for their use in size-selecting nucleic acids. Thus, SPRI beads can be used to selectively enrich for nucleic acid molecules or fragments of a specified length from a sample, or fragments having a length shorter or longer than a specified cut-off length (e.g. >5 kb, ≥7.5 kb or ≥10 kb in length as described herein). In size-selection applications, the concentration of crowding agent and the ratio of SPRI beads in binding buffer to sample is critical and influence the size/length of nucleic acid molecules/fragments being selected. As a general rule, the lower the ratio of SPRI beads to sample (e.g. SPRI:DNA), the larger/longer the selected fragments. Conversely, the higher the ratio/volume of SPRI beads in binding buffer added to the sample, the smaller/shorter the length cut-off for selected nucleic acid fragments. For example, it has been shown herein that a ratio of SPRI beads in binding buffer to sample of 0.97 (i.e. 0.97× volume of SPRI beads in binding buffer are added to 1× volume of sample) provides accurate and specific selection of nucleic acid fragments greater than 5 kb in length, while a ratio of SPRI beads in binding buffer to sample of 0.94 (i.e. 0.94× volume of SPRI beads in binding buffer are added to 1× volume of sample) provides accurate and specific selection of nucleic acid fragments greater than 7.5 kb in length. Thus, the method described herein provides accurate and specific removal of nucleic acid fragments 5 kb or less in length from a fragmented nucleic acid sample when a ratio of SPRI beads in binding buffer to sample of 0.97× is used, and accurate and specific removal of nucleic acid fragments 7.5 kb or less in length when a ratio of 0.94× is used. The method also provides accurate and specific removal of nucleic acid fragments 5 kb or less in length when a ratio of SPRI beads in binding buffer to sample of 0.96× is used. It will be appreciated that references herein to ratios refer to volumetric ratios (i.e. v:v or v/v) of the volume of SPRI beads in binding buffer to the volume of sample, such as fragmented nucleic acid sample (e.g. SPRI:DNA). Ratios may also be expressed in the form of a multiplier (i.e. “×”), where the amount/volume of SPRI beads in binding buffer is expressed relative to the amount/volume of fragmented nucleic acid sample (wherein the amount/volume of sample is 1×). In one embodiment, the ratio of SPRI beads in binding buffer to sample is between 0.97× and 0.91× (wherein the end values are included in said range). In another embodiment, the ratio of SPRI beads in binding buffer to sample is between 0.97× and 0.94× (wherein the end values are included in said range). In a further embodiment, the ratio of SPRI beads in binding buffer to sample is 0.97×. In a yet further embodiment, the method comprises selecting for nucleic acid fragments greater than about 5 kb in length and the ratio of SPRI beads in binding buffer to sample is 0.97×. In a still further embodiment, selecting nucleic acid fragments removes fragments about 5 kb or less in length from the fragmented nucleic acid sample and the ratio of SPRI beads in binding buffer to sample is 0.97×. In a further embodiment, the ratio of SPRI beads in binding buffer to sample is 0.96×. In a yet further embodiment, the method comprises selecting for nucleic acid fragments greater than about 5 kb in length and the ratio of SPRI beads in binding buffer to sample is 0.96×. In a still further embodiment, selecting nucleic acid fragments removes fragments about 5 kb or less in length from the fragmented nucleic acid sample and the ratio of SPRI beads in binding buffer to sample is 0.96×. In another embodiment, the ratio of SPRI beads in binding buffer to sample is 0.94×. In a further embodiment, the method comprises selecting for nucleic acid fragments greater than about 7.5 kb in length (e.g. fragments about 8 kb or greater in length) and the ratio of SPRI beads in binding buffer to sample is 0.94×. In a yet further embodiment, selecting nucleic acid fragments removes fragments about 7.5 kb or less in length (e.g. fragments less than about 8 kb in length) from the fragmented nucleic acid sample and the ratio of SPRI beads in binding buffer to sample is 0.94×. In another embodiment, the ratio of SPRI beads in binding buffer to sample is 0.91×. In a further embodiment, the method comprises selecting for nucleic acid fragments about 9 kb or greater in length and the ratio of SPRI beads in binding buffer to sample is 0.91×. In a yet further embodiment, selecting nucleic acid fragments removes fragments less than about 9 kb in length from the fragmented nucleic acid sample and the ratio of SPRI beads in binding buffer to sample is 0.91×. In particular embodiments, the ratio of SPRI beads in binding buffer to sample is exactly 0.97×, 0.96×, 0.94×or 0.91×.

### Binding Buffer

In one embodiment, the binding buffer comprises: PEG6000 (also known as PEG6k) at a concentration of 10%; NaCl (sodium chloride salt) at a concentration of 1900 mM; and TrisHCl pH8 at a concentration of 10 mM, i.e. the binding buffer comprises 10% PEG6000, 1900 mM NaCl and 10 mM TrisHCl pH8. This binding buffer composition has been shown herein to provide accurate and specific selection for nucleic acid fragments of the specified size when used with SPRI beads at the appropriate volumetric ratio to a sample (e.g. a fragmented nucleic acid sample). Such selection efficiently and accurately removes nucleic acid fragments having lengths/sizes other than the specified length, such as those shorter/having a length less than the specified selection length. For example, wherein SPRI beads in the binding buffer described herein are used at a ratio to sample of 0.97×, nucleic acid fragments greater than 5 kb in length are efficiently and accurately/specifically selected for, while fragments 5 kb or less in length are efficiently and accurately/specifically removed from the generated nucleic acid library (see FIGS. 1-7). In a further example, wherein SPRI beads in the binding buffer described herein are used a ratio to sample of 0.96×, nucleic acid fragments greater than 5 kb in length are efficiently and accurately/specifically selected for, while fragments 5 kb or less in length are efficiently and accurately/specifically removed from the generated nucleic acid library (see FIG. 1, bottom panel). In a yet further example, wherein SPRI beads in the binding buffer described herein are used at a ratio to sample of 0.94×, nucleic acid fragments greater than 7.5 kb in length are efficiently and accurately/specifically selected for, while fragments 7.5 kb or less in length are efficiently and accurately/specifically removed from the generated nucleic acid library. Furthermore, use of the binding buffer described herein provides high recovery/yield of the selected nucleic acid fragments from the sample, such that those nucleic acid fragments of the specified size present in the sample are retained in the nucleic acid library as described hereinbefore. For example, the recovery of selected nucleic acid fragments in the generated nucleic acid library may be 51-100% or 42-100% of the nucleic acid fragments of the specified length in the fragmented nucleic acid sample.

In some embodiments, the binding buffer comprises: PEG6000 at a final concentration of 4.92%; NaCl at a final concentration of 939 mM; and TrisHCl pH8 at a final concentration of 4.92 mM. The term “final concentration” as used herein refers to the concentration of the recited binding buffer component in the final binding reaction mixture, i.e. following addition of the binding buffer containing SPRI beads to a sample. Thus, when calculating final concentration, the volumetric ratio/amount of SPRI beads in binding buffer to be added to the fragmented nucleic acid sample must be taken into consideration.

In a further embodiment, the binding buffer additionally comprises 1 mM EDTA. Thus, in some embodiments, the binding buffer comprises EDTA at a final concentration of 0.49 mM.

### Sequencing

In one embodiment, the nucleic acid library is for long-read sequencing, such as Single Molecule, Real-Time (SMRT) sequencing. Long-read sequencing (also known as third generation sequencing) techniques provide the capability to produce long sequence reads which are substantially longer than those generated by previous generation sequencing (e.g. second generation or next generation sequencing (NGS)). Similar to NGS, long-read/third generation sequencing may be a sequencing-by-synthesis technique using a polymerase. Examples of NGS sequencing-by-synthesis platforms include Illumina's TruSeq technology which supports massively parallel sequencing (MPSS; massively parallel signature sequencing) using a proprietary reversible terminator-based method that enables detection of single bases as they are incorporated into growing DNA strands, as well as the Roche 454 platform (i.e. Roche 454 GS FLX) which employs pyrosequencing, whereby a chemiluminescent signal indicates base incorporation and the intensity of signal correlates to the number of bases incorporated through homopolymer reads; Applied Biosystems'SOLID system (i.e. SOLIDv4); Illumina's GAIIx, HiSeq 2000, NovaSeq, HiSeq4000 and MiSeq sequencers; Life Technologies'Ion Torrent semiconductor-based sequencing instruments which use a strategy similar to sequencing-by-synthesis but detect signal by the release of hydrogen ions resulting from the activity of DNA polymerase during nucleotide incorporation; Pacific Biosciences'PacBio RS; Sanger's 3730xl; and nanopore-based sequencing platforms such as those in which the nanopore is constructed from a metal, polymer or plastic material or Oxford Nanopore Technologies'organic-type nanopore-based system which mimics the situation of the cell membrane and protein channels in living cells.

Long-read sequencing methods include Single Molecule, Real-Time (SMRT) sequencing from PacBio and nanopore sequencing from Oxford Nanopore. SMRT sequencing is based on the sequencing-by-synthesis approach and uses zero-mode wave-guides (ZMWs; small well-like containers with capturing tools located at the bottom of the well) in which synthesis occurs. During sequencing each ZMW contains a single polymerase and a single nucleic acid template strand attached to the bottom of the ZMW, and the ZMWs have properties and are of a dimension (approx. 70 nm in diameter and approx. 100 nm in depth) that only fluorescence occurring at the very bottom of the well is detected (the observation volume in each ZMW is approx. 20 zeptolitres). Upon incorporation of a fluorescently labelled nucleotide into the synthesised strand, the fluorescent tag is cleaved and diffuses out of the observation area of the ZMW meaning that its fluorescence is no longer detectable. This is measured by the sequencer and the identity of the incorporated nucleotide is called. Modifications in the template nucleic acid strand can be detected by measuring the kinetics of nucleotide incorporation and the polymerase. The method described herein finds particular utility in generating a nucleic acid library for SMRT sequencing, such as on a PacBio sequencer. Nanopore sequencing measures the change in ion current as a nucleic acid is passed through a nanopore and does not rely on sequencing-by-synthesis. This change is dependent on the shape, size and length of the nucleic acid strand and the period of time that each nucleotide blocks ion flow through the pore varies according to the identity of the nucleotide. Measurement is in real-time and no modified or labelled nucleotides are required. In some embodiments, the long-read sequencing is circular consensus sequencing (CCS) and/or continuous long read (CLR) sequencing. CCS generates consensus sequences from multiple passes around a single circularised nucleic acid molecule. The multiple passes (also known as sub-reads) are then used to calculate the consensus sequence of the template. The consensus sequence can be used with barcode sequences in multiplexed samples and for mapping to a reference sequence, such as a reference genome. Continuous long read (CLR) sequencing uses a consensus sequence called from multiple sub-reads of a long sequence read to generate highly accurate sequence data with reads up to 25 kb in length (known as HiFi reads). Both CCS and CLR sequencing utilise SMRT sequencing and can be performed on PacBio sequencers, such as the Sequel, Sequel II and Sequel IIe systems.

The generation of a nucleic acid library for sequencing as described herein may further comprise adding sequencing adaptors to the nucleic acid fragments. Therefore, in one embodiment, the method comprises the step of adding sequencing adaptors to the selected nucleic acid fragments, i.e. the sequencing adaptors are added following selecting nucleic acid fragments of a specified size. In another embodiment, the method comprises the step of adding sequencing adaptors to the fragmented nucleic acid sample prior to selecting nucleic acid fragments of a specified size (e.g. greater than 5 kb in length, such as ≥7.5 kb in length), i.e. the sequencing adaptors are added to the fragmented nucleic acid sample. Such sequencing adaptors allow automated high throughput sequencing, such as NGS and/or long-read sequencing as described hereinbefore. For example, the sequencing adaptors may circularise the selected nucleic acid fragments to allow SMRT sequencing, such as CCS and/or CLR sequencing. Thus, in one embodiment, the method comprises adding sequencing adaptors to circularise the selected nucleic acid fragments. Such adaptors include, for example, the SMRTbell adaptors from PacBio. In a further embodiment, the sequencing adaptors comprise a barcode sequence. Barcode sequences are used to identify sequencing reads originating from a single sample when multiple samples are multiplexed.

In one embodiment, the method of generating a nucleic acid library for sequencing further comprises the step of end-repairing the fragmented nucleic acid sample and/or selected nucleic acid fragments. Such end-repair is performed prior to any optional addition of sequencing adaptors as described hereinbefore and may comprise removing single-stranded overhangs from the fragmented nucleic acid sample and/or the ‘filling in’ of single-stranded regions of the nucleic acid sample/fragments by copying 5′ overhangs to generate blunt-ended nucleic acid fragments. In one embodiment, end-repair is performed prior to selecting nucleic acid fragments of a specified size (e.g. greater than 5 kb in length, such as ≥7.5 kb in length). In an alternative embodiment, end-repair is performed following selecting nucleic acid fragments of a specified size. In another embodiment, end-repair is performed prior to and following selecting nucleic acid fragments of a specified size. Following end-repair, the nucleic acid fragments (e.g. the selected nucleic acid fragments or the fragmented nucleic acid sample) are A-tailed.

In some embodiments, the SPRI beads may require washing prior to use for selecting nucleic acid fragments of a specified size, depending on the sequencing technology being used to sequence the nucleic acid library. In certain embodiments, the SPRI beads may require extensive washing. For example, if AMPure XP SPRI beads (Beckman Coulter) are used for selecting nucleic acid fragments and the nucleic acid library is analysed on a PacBio sequencer, up to 5 washes may be required. Thus, in one embodiment, the SPRI beads are washed 5 times prior to selecting nucleic acid fragments. In a further embodiment, the SPRI beads are prepared by washing 5 times. In a yet further embodiment, the SPRI beads are washed (e.g. 5 times) using water, such as nuclease free water and/or molecular biology grade water. In another embodiment, the SPRI beads are washed 2 times during selection of nucleic acid fragments, such as following immobilisation/binding of nucleic acid fragments on the SPRI beads and prior to addition of an aqueous elution buffer. Thus, in a yet further embodiment, the SPRI bead/nucleic acid fragment complexes are washed, such as washed 2 times. According to this embodiment, when the SPRI bead/nucleic acid fragment complexes are washed during selection, the washes are performed in the presence of a magnetic field. Still according to this embodiment, the SPRI bead/nucleic acid fragment complexes are washed using ethanol (EtOH). In a further embodiment, a final wash of the SPRI beads in the binding buffer described herein is performed prior to selection, i.e. prior to addition of the SPRI beads in binding buffer to the fragmented nucleic acid sample and prior to immobilisation/binding of nucleic acid fragments on the SPRI beads.

Therefore, in an exemplary embodiment, the SPRI beads are prepared prior to selecting nucleic acid fragments, said preparation comprising the steps described in Example 1.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between”as used herein includes the values of the specified boundaries.

It will be understood that all embodiments described herein may be applied to all aspects of the invention and vice versa. It will be further understood that the method and any/all steps thereof described herein are performed in vitro or on ex vivo samples.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The invention will now be described using the following, non-limiting examples:

### EXAMPLES

### Example 1—Preparation of SPRI Beads

Prior to selecting nucleic acid fragments (i.e. prior to addition of the SPRI beads in binding buffer to the fragmented nucleic acid sample and prior to immobilisation/binding of nucleic acid fragments on the SPRI beads), the SPRI beads may be prepared according to the following steps:
1. thoroughly resuspending SPRI beads, such as AMPure Xp beads (Beckman Coulter);
2. transferring 500 μl of SPRI beads into a 1.5 mL microtube;
3. pelleting SPRI beads by centrifuging for 1 minute at 16 k rpm (or maximum speed) in a bench top microcentrifuge;
4. placing the tube on a magnet rack (i.e. in the presence of a magnetic field);
5. removing and discarding supernatant;
6. adding 1 mL molecular biology grade water to the beads, and resuspending beads completely by vortexing;
7. pelleting the beads by centrifuging for 1 minute at 16 k rpm (or maximum speed) in a bench top microcentrifuge;
8. placing the tube on a magnet rack (i.e. in the presence of a magnetic field);
9. removing and discarding water wash;
10. repeating water wash 4 more times (steps 6 to 8);
11. adding 1 mL buffer EB (Qiagen) to beads, and resuspending beads completely by vortexing;
12. pelleting the beads by centrifuging for 1 minute at 16 k rpm (or maximum speed) in a bench top microcentrifuge;

13. placing the tube on a magnet rack (i.e. in the presence of a magnetic field);
14. removing and discarding Tris (buffer EB) wash; and
15. resuspending beads in binding buffer (100 μl), placing on magnet (i.e. in presence of magnetic field), removing supernatant and resuspending beads in binding buffer (500μl).

Preparation of SPRI beads according to these steps finds particular utility when the generated nucleic acid library is analysed using a PacBio sequencer. According to a specific example, the SPRI beads used for selecting nucleic acid fragments are AMPure XP SPRI beads (Beckman Coulter) and the nucleic acid library is analysed by SMRT sequencing on a PacBio sequencer, such as the Sequel, Sequel II or Sequel IIe system.

### Example 2—Selecting Nucleic Acid Fragments Greater Than 7.5 Kb in Length

Selecting nucleic acid fragments from a fragmented nucleic acid sample using SPRI beads in the binding buffer described herein (i.e. immobilising/binding nucleic acid fragments of a specified size on the SPRI beads), wherein the selected nucleic acid fragments are greater than 7.5 kb in length (i.e. using a ratio of SPRI beads in binding buffer to sample of 0.97×), may be performed according to the following steps:
1. top up sample to 101 μl, and accurately transfer 100 μl to a fresh tube (to ensure accuracy and that exactly 100 μl is transferred, the tip may be pre-wetted by pipetting up and down twice and then dispensing into the new tube to the stop point of the pipette);
2. bring the SPRI beads in binding buffer to room temperature and mix thoroughly by vortexing;
3. aspirate 97 μl of the SPRI beads in binding buffer by putting pipette tip just to the top of the liquid level (so extra volume is not adhered to the outside of the tip), aspirate once, dispense into the sample and mix by pipetting up and down twice (using the same pipette throughout because it's calibrated exactly the same);
4. mix 10 times by pipetting with a wide-bore tip;
5. incubate on a rotator/hula mixer at a very low speed for 30 minutes to immobilise/bind the nucleic acid fragments on the SPRI beads);
6. place SPRI beads in binding buffer/sample mix on a magnet (i.e. in a magnetic field) and wash twice with 80% EtOH;
7. elute in desired amount of elution buffer (EB) for 15 minutes at 37° C., and transfer eluate (containing the selected nucleic acid fragments) to fresh tube.

Extra pipetting during the above steps should not be detrimental to the fragment size (e.g. by causing shearing/further shearing) due to the size of fragments being selected for (>7.5 kb in length). The mixing by pipetting described is highly beneficial to the size selection, e.g. by ensuring tip contents have been completely washed out and the anticipated/desired volume has been transferred. However, unnecessary pipetting/unnecessarily forceful pipetting should be avoided to ensure that the length of fragments is maintained.

The results of an example selection of nucleic acid fragments using the above method are shown in FIG. 1. gDNA containing a broad smear of fragment sizes was used for size selection. Prior to selection many nucleic acid fragments <10 kb, <7.5 kb and <5 kb in length can be detected in the sample (FIG. 1, top panel), while following selection using SPRI beads in binding buffer as described herein at ratios to sample of 0.91×, 0.94×, 0.96×, 0.97× or 0.91×, or using AMPure XP beads at a ratio of 0.36× (FIG. 1, bottom panel) selects for fragments great than 5 kb in length, such as fragments greater than 7.5 kb in length. Fragments greater than 10 kb in length are also substantially selected.

### Example 3—Sequencing of Mouse gDNA

FIG. 2A shows the size analysis of gDNA from mouse subjected to the selection of nucleic acid fragments of a specified size as described herein. Prior to selection 10% of the molar amount of nucleic acid fragments are <5 kb in length and the average length of nucleic acid fragments in the sample is <16 kb (FIG. 2A, bottom trace). Following selection the average size of nucleic acid fragments in the sample increases to >16 kb (FIG. 2A, middle and top traces) and fragments ≤5 kb in length have been removed (FIG. 2A, top trace).

The selected nucleic acid fragments in FIG. 2A were sequenced and the read length distribution analysed (FIG. 2B). Thus, the selected nucleic acid fragments can be used to generate a nucleic acid library from mouse for sequencing which yields sequencing reads enriched for those >5 kb in length, such as ≥7.5 kb in length, due to a substantial reduction of reads ≤5 kb in length, such as reads <7.5 kb in length.

### Example 4—Sequencing of Mistletoe gDNA

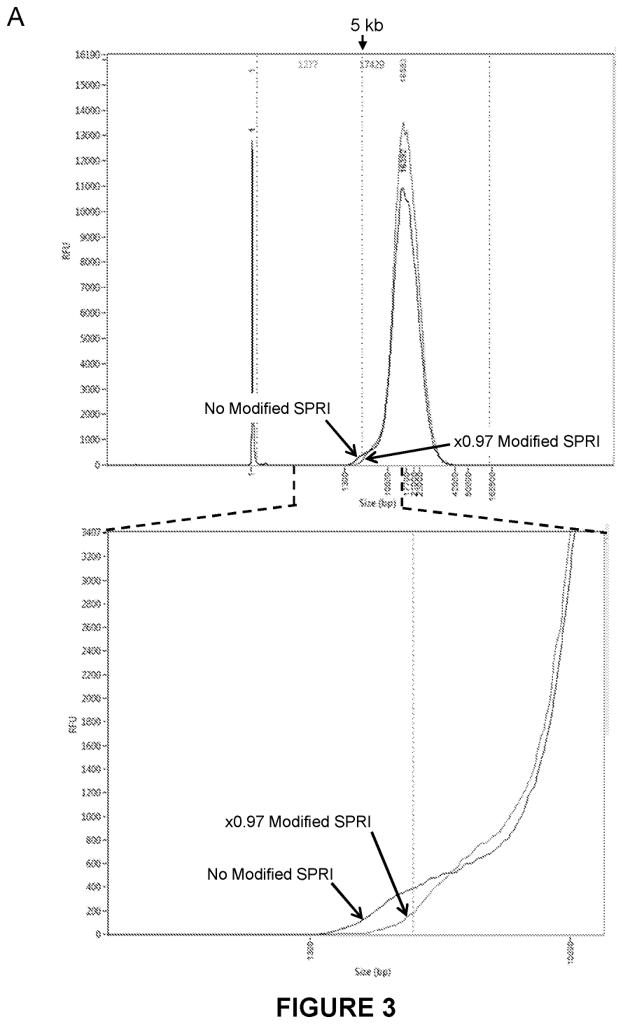

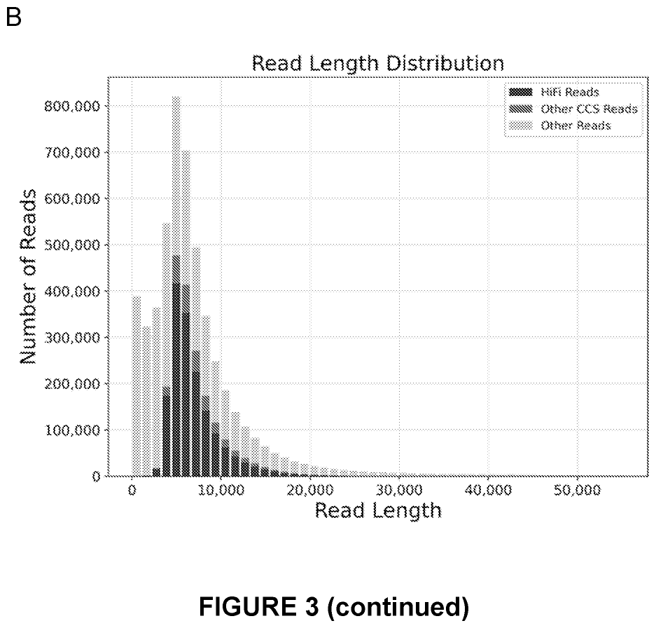

A fragmented nucleic acid sample containing gDNA from mistletoe was analysed in the same way as described in Example 3 (FIG. 3). As shown in FIG. 3A, substantially all nucleic acid fragments 5 kb or less in length are removed following selection (FIG. 3A, bottom trace compared to top trace (without selection)).

The selected nucleic acid fragments in FIG. 3A were sequenced and the read length distribution analysed (FIG. 3B). Thus, the selected nucleic acid fragments can be used to generate a nucleic acid library from mistletoe for sequencing which yields sequencing reads enriched for those >5 kb in length, such as ≥7.5 kb in length, due to a substantial reduction of reads ≤5 kb in length, such as reads <7.5 kb in length.

### Example 5—Sequencing of Green Algae gDNA

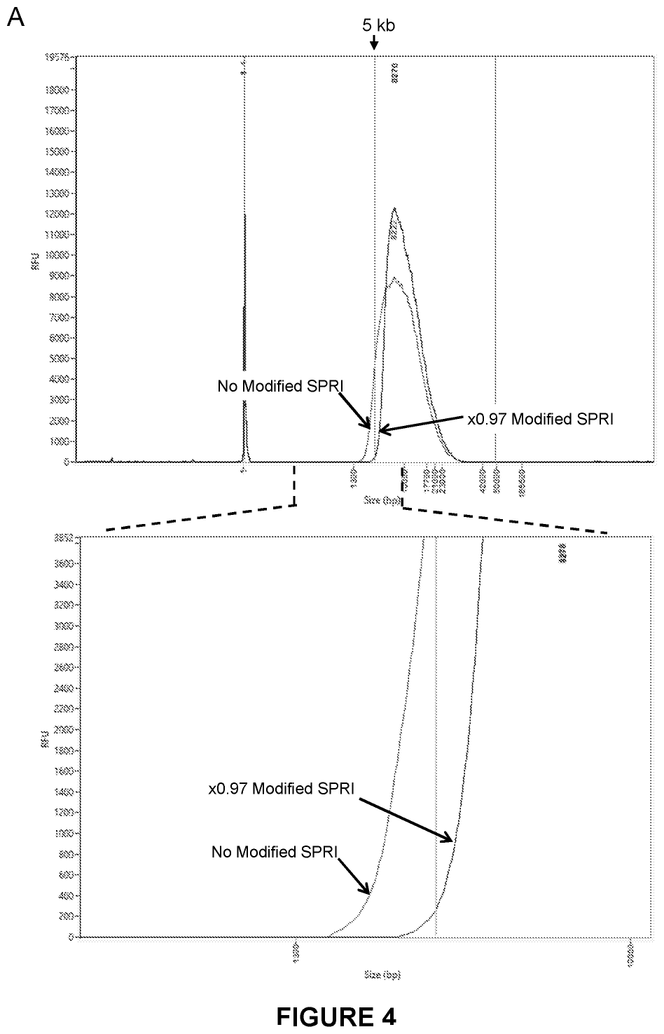

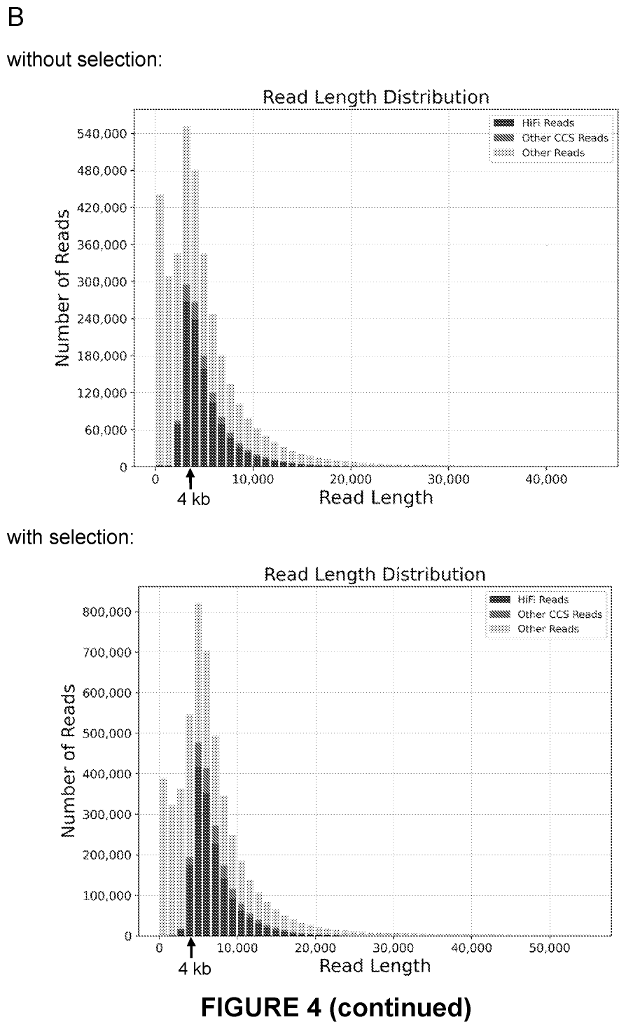

A fragmented nucleic acid sample containing gDNA from green algae was analysed in the same way as described in Example 3 (FIG. 4). As shown in FIG. 4A, substantially all nucleic acid fragments 5 kb or less in length are removed following selection (FIG. 4A, bottom trace compared to top trace (without selection)).

The selected nucleic acid fragments in FIG. 4A were sequenced and the read length distribution analysed (FIG. 4B). Thus, the selected nucleic acid fragments can be used to generate a nucleic acid library from green algae for sequencing which yields sequencing reads enriched for those >5 kb in length, such as ≥7.5 kb in length, due to a substantial reduction of reads ≤5 kb in length, such as reads <7.5 kb in length.

### Example 6—Sequencing of Snail gDNA

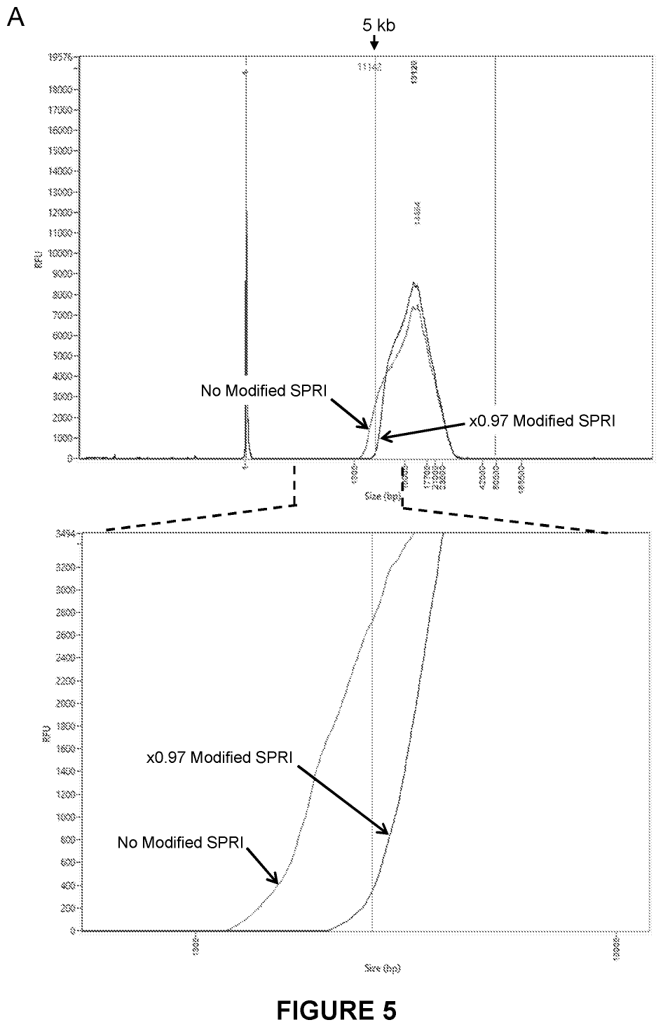

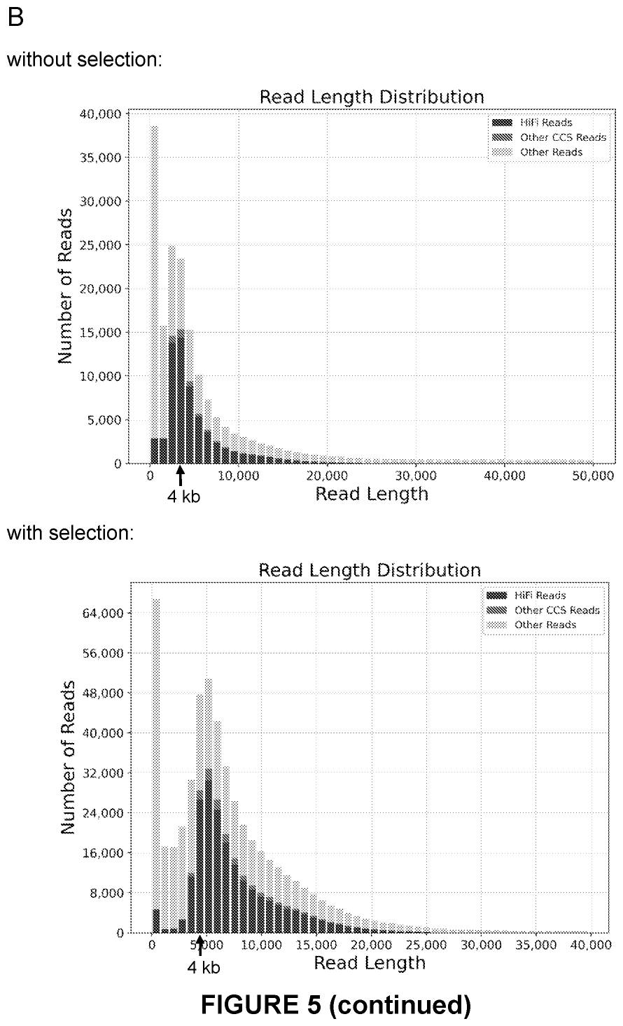

A fragmented nucleic acid sample containing gDNA from snail was analysed in the same way as described in Example 3 (FIG. 5). As shown in FIG. 5A, substantially all nucleic acid fragments 5 kb or less in length are removed following selection (FIG. 5A, bottom trace compared to top trace (without selection)).

The selected nucleic acid fragments in FIG. 5A were sequenced and the read length distribution analysed (FIG. 5B). Thus, the selected nucleic acid fragments can be used to generate a nucleic acid library from snail for sequencing which yields sequencing reads enriched for those >5 kb in length, such as ≥7.5 kb in length, due to a substantial reduction of reads ≤5 kb in length, such as reads <7.5 kb in length.

### Example 7—Sequencing of Ladybird gDNA

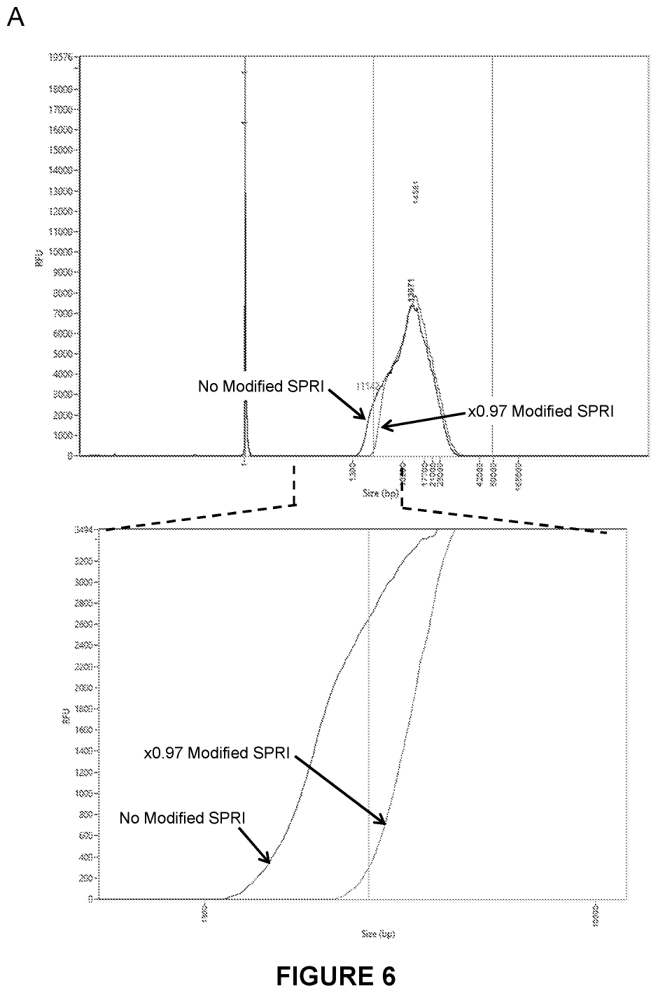

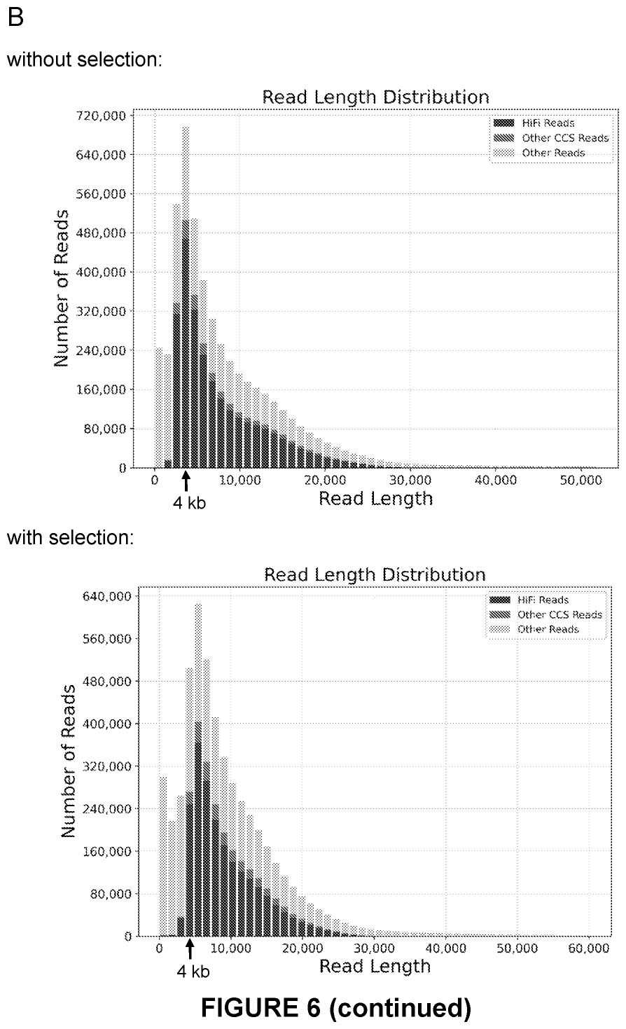

A fragmented nucleic acid sample containing gDNA from ladybird was analysed in the same way as described in Example 3 (FIG. 6). As shown in FIG. 6A, substantially all nucleic acid fragments 5 kb or less in length are removed following selection (FIG. 6A, bottom trace compared to top trace (without selection)).

The selected nucleic acid fragments in FIG. 6A were sequenced and the read length distribution analysed (FIG. 6B). Thus, the selected nucleic acid fragments can be used to generate a nucleic acid library from ladybird for sequencing which yields sequencing reads enriched for those >5 kb in length, such as ≥7.5 kb in length, due to a substantial reduction of reads ≤5 kb in length, such as reads <7.5 kb in length.

### Example 8—Sequencing of Oak Tree gDNA

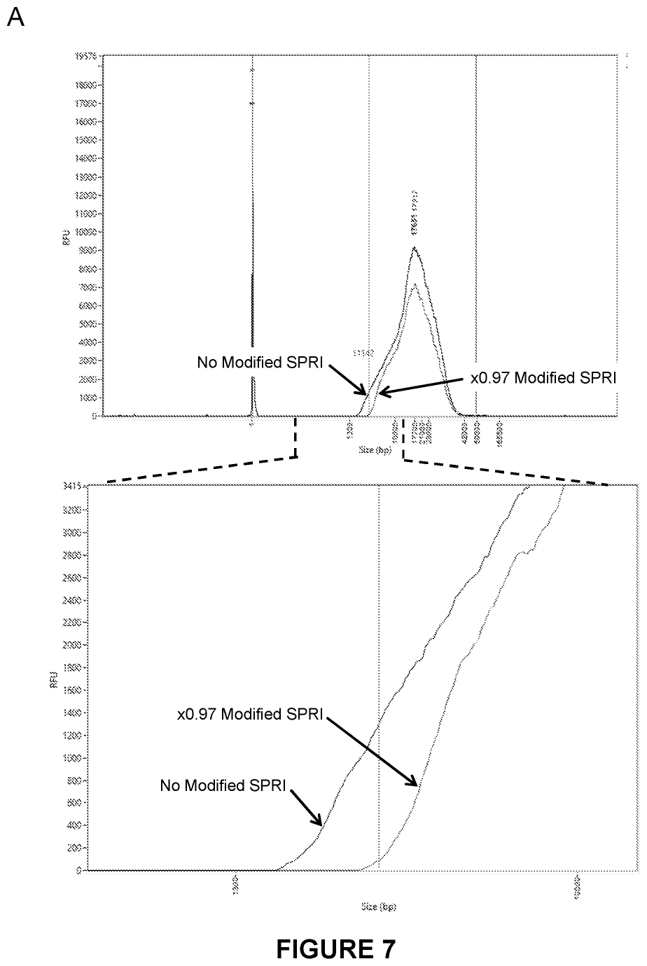

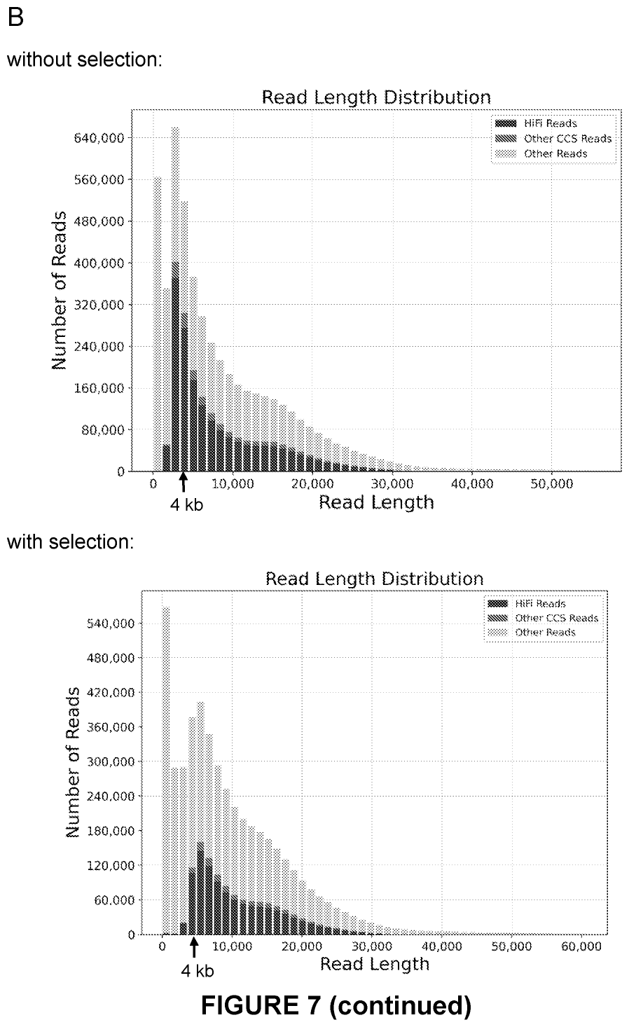

A fragmented nucleic acid sample containing gDNA from mistletoe was analysed in the same way as described in Example 3 (FIG. 7). As shown in FIG. 7A, substantially all nucleic acid fragments 5 kb or less in length are removed following selection (FIG. 7A, bottom trace compared to top trace (without selection)).

The selected nucleic acid fragments in FIG. 7A were sequenced and the read length distribution analysed (FIG. 7B). Thus, the selected nucleic acid fragments can be used to generate a nucleic acid library from oak tree for sequencing which yields sequencing reads enriched for those >5 kb in length, such as ≥7.5 kb in length, due to a substantial reduction of reads ≤5 kb in length, such as reads <7.5 kb in length.

1. A method of generating a nucleic acid library for sequencing, said method comprising selecting for nucleic acid fragments greater than 5 kb in length from a fragmented nucleic acid sample using paramagnetic Solid Phase Reversible Immobilization (SPRI) beads in a binding buffer comprising: 10% PEG6000; 1900 mM NaCl; and 10 mM TrisHCl pH8,
wherein the ratio of SPRI beads in binding buffer to sample is between 0.97× and 0.91×.

2. The method of claim 1, wherein selecting nucleic acid fragments removes nucleic acid fragments 5 kb of less in length from the fragmented nucleic acid sample.

3. The method of claim wherein the selected nucleic acid fragments are greater than or equal to 7.5 kb in length.

4. The method of claim 3, wherein selecting nucleic acid fragments removes nucleic acid fragments 7.5 kb or less in length from the fragmented nucleic acid sample.

5. The method of claim 1, wherein the selected nucleic acid fragments are greater than or equal to 10 kb in length.

6. The method of claim 5, wherein selecting nucleic acid fragments removes nucleic acid fragments 10 kb or less in length from the fragmented nucleic acid sample.

7. The method of claim 1, wherein the ratio of SPRI beads in binding buffer to sample is 0.94×.

8. The method of claim 1, wherein the ratio of SPRI beads in binding buffer to sample is 0.96×or 0.97×.

9. The method of claim 1, wherein the selected nucleic acid fragments are greater than 5 kb in length and the ratio of SPRI beads in binding buffer to sample is 0.97×.

10. The method of claim 1, wherein the selected nucleic acid fragments are greater than 5 kb in length and the ratio of SPRI beads in binding buffer to sample is 0.96×.

11. The method of claim 1, wherein the selected nucleic acid fragments are greater than or equal to 7.5 kb in length and the ratio of SPRI beads in binding buffer to sample is 0.94×.

12. The method of claim 1, to wherein the generated nucleic acid library comprises nucleic acid fragments greater than 42 kb in length.

13. The method of claim 1, wherein the fragmented nucleic acid sample comprises genomic DNA (gDNA).

14. The method of claim 1, to wherein the nucleic acid library is for long-read sequencing, such as Single Molecule, Real-Time (SMRT) sequencing.

15. The method of claim 14, wherein the long-read sequencing is circular consensus sequencing (CCS) and/or continuous long read (CLR) sequencing.

16. The method of claim 14, wherein the SPRI beads are washed 5 times prior to selecting nucleic acid fragments.

17. The method of claim 14, wherein a final wash of the SPRI beads in binding buffer is performed prior to selecting nucleic acid fragments.

18. A method of sequencing the genome of an organism, comprising performing the method of generating a nucleic acid library according to claim 1.

19. The method of claim 18, wherein the genome is a novel genome.

20. The method of claim 18, wherein the method is for generating a novel genome assembly.
